(19) United States
(12) Patent Application Publication
(10) Pub. No.: US 2015/0105735 A1
(43) Pub. Date: Apr. 16, 2015

(54) SYRINGE PISTONS, SYSTEMS AND METHODS

(71) Applicant: REGENERON PHARMACEUTICALS, INC., Tarrytown, NY (US)

(72) Inventors: Michael S. MARUK, Pittsfield, MA (US); Bernard ETTINGER, Glens Falls, NY (US); Joseph BERRY, Neshanic Station, NJ (US); Alexei GORALTCHOUK, Rensselaer, NY (US); Mykhaylo HRYTSAK, Hastings on Hudson, NY (US)

(73) Assignee: REGENERON PHARMACEUTICALS, INC., Tarrytown, NY (US)

(21) Appl. No.: 14/516,322

(22) Filed: Oct. 16, 2014

Related U.S. Application Data
(60) Provisional application No. 61/891,519, filed on Oct. 16, 2013.

(51) Int. Cl. A61M 5/315

(52) U.S. Cl. CPC A61M 5/31511
USPC 604/218; 92/240; 92/242

(57) ABSTRACT

Pistons for a syringe and syringe systems are disclosed. The pistons may include a base with a first end, a second end opposite the first end, and a side portion extending between the first end and the second end. The piston may also include at least one engagement portion extending around the circumference of the side portion of the base. Another embodiment of the plunger head may include a proximal end, a distal end opposite the proximal end, and an intermediate portion connecting the proximal end and the distal end. The intermediate portion is tapered from the proximal end to the distal end. The syringe system includes a piston and a plunger rod. The plunger rod may include a first end and a second end with the first end pressingly contacting the distal end of the piston.

### CROSS REFERENCE TO RELATED APPLICATION

This application claims priority benefit under 35 U.S.C. §119(e) of U.S. provisional application No. 61/891,519 filed Oct. 16, 2013, which is incorporated herein by reference in its entirety.

### FIELD OF THE INVENTION

The present invention relates generally to auto injector syringe for administering medication. More specifically, but not exclusively, the present invention concerns pistons useful in syringes, such as, auto injector syringes.

### BACKGROUND OF THE INVENTION

Currently available syringes, including auto injector syringes, may experience variable friction forces which contribute to inconsistent injection times. In addition, current syringes have variable glide forces that require different amounts of pressure to be applied on the syringe plungers during injection. The application of different pressures during injection may result in inconsistent amounts of medication being injected into the patient. The manufacturing of currently available syringes and pistons may be costly and difficult. Currently, it is difficult to machine fill-finish lines of auto injector syringes because the pistons are dimensionally inconsistent. The existing piston designs are manufactured with forming and cutting steps that result in sidewall variation which may be due to one or both of the material and cutting process being misaligned resulting in asymmetrical piston geometries.

### SUMMARY OF THE INVENTION

Aspects of the present invention provide syringe pistons, systems and methods for use in injecting medication into a patient.

In one aspect provided herein is a piston for a syringe including a base with a first end, a second end opposite the first end, and a side portion extending between the first end and the second end. The piston may also include at least one engagement portion extending around the circumference of the side portion of the base.

In another aspect, provided herein is a plunger head for a syringe that includes a proximal end, a distal end opposite the proximal end, and an intermediate portion connecting the proximal end and the distal end. The intermediate portion is tapered from the proximal end to the distal end.

In yet another aspect provided herein is a syringe system that has a piston and a plunger rod. The piston includes a proximal end, a distal end, a side section extending between the proximal end and the distal end, and at least one engagement portion extending around the circumference of the side section of the base and away from the side section. The proximal end is shaped for engagement with a medication located in a syringe chamber. The at least one engagement portion is dimensioned to engage the syringe chamber. The plunger rod also includes a first end and a second end with the first end pressingly contacting the distal end of the piston.

These, and other objects, features and advantages of this invention will become apparent from the following detailed description of the various aspects of the invention taken in conjunction with the accompanying drawings.

### BRIEF DESCRIPTION OF DRAWINGS

The accompanying drawings, which are incorporated in and constitute a part of the specification, illustrate embodiments of the invention and together with the detailed description herein, serve to explain the principles of the invention. The drawings are only for purposes of illustrating preferred embodiments and are not to be construed as limiting the invention. It is emphasized that, in accordance with the standard practice in the industry, various features are not drawn to scale. In fact, the dimensions of the various features may be arbitrarily increased or reduced for clarity of discussion. The foregoing and other objects, features and advantages of the invention are apparent from the following detailed description taken in conjunction with the accompanying drawings in which:

FIG. 1 is a cross section of an embodiment of a syringe system with a piston and plunger rod, in accordance with an aspect of the present invention;

FIG. 2 is a perspective side view of the piston of FIG. 1, in accordance with an aspect of the present invention;

FIG. 3 is a side view of the piston of FIG. 1, in accordance with an aspect of the present invention;

FIG. 4 is cross sectional view of another embodiment piston, in accordance with an aspect of the present invention;

FIG. 5 is a perspective view of the piston of FIG. 4, in accordance with an aspect of the present invention;

FIG. 6 is a side view of the piston of FIG. 4, in accordance with an aspect of the present invention;

FIG. 7 is a cross sectional view of an embodiment of a piston showing alternative distal ends, in accordance with an aspect of the present invention;

FIG. 8 is a perspective side view of the piston of FIG. 7, in accordance with an aspect of the present invention;

FIG. 9 is a side view of the piston of FIG. 7, in accordance with an aspect of the present invention;

FIG. 10 is a perspective bottom view of an embodiment of the piston of FIG. 7, in accordance with an aspect of the present invention;

FIG. 11 is a perspective bottom view of another embodiment of the piston of FIG. 7, in accordance with an aspect of the present invention;

FIG. 12 is a cross sectional view of another embodiment syringe system with a piston and a plunger rod, in accordance with an aspect of the present invention;

FIG. 13 is a perspective side view of the piston of FIG. 12, in accordance with an aspect of the present invention;

FIG. 14 is a perspective bottom view of the piston of FIG. 12, in accordance with an aspect of the present invention;

FIG. 15 is a side view of the piston of FIG. 12, in accordance with an aspect of the present invention;

FIG. 16 is a side view of another embodiment of a piston, in accordance with an aspect of the present invention;

FIG. 17 is a perspective view of the piston of FIG. 16, in accordance with an aspect of the present invention; and

FIG. 18 is a perspective side view of the piston of FIG. 16 with a portion removed to show the cross section, in accordance with an aspect of the present invention.

### DETAILED DESCRIPTION FOR CARRYING OUT THE INVENTION

Generally stated, disclosed herein are a number of embodiments of pistons and plunger rods. In addition, syringe systems incorporating the pistons are disclosed. Further, a method of manufacturing the pistons is discussed.

In this detailed description and the following claims, the words proximal, distal, anterior, posterior, medial, lateral, superior and inferior are defined by their standard usage for indicating a particular part of a device according to the relative disposition of the device with respect to a body or directional terms of reference. For example, “proximal” means the portion of a device nearest the point of attachment, while “distal” indicates the portion of the device farthest from the point of attachment. As for directional terms, “anterior” is a direction towards the front side of the device, “posterior” means a direction towards the back side of the device, “medial” means towards the midline of the device, “lateral” is a direction towards the sides or away from the midline of the device, “superior” means a direction above and “inferior” means a direction below another object or structure.

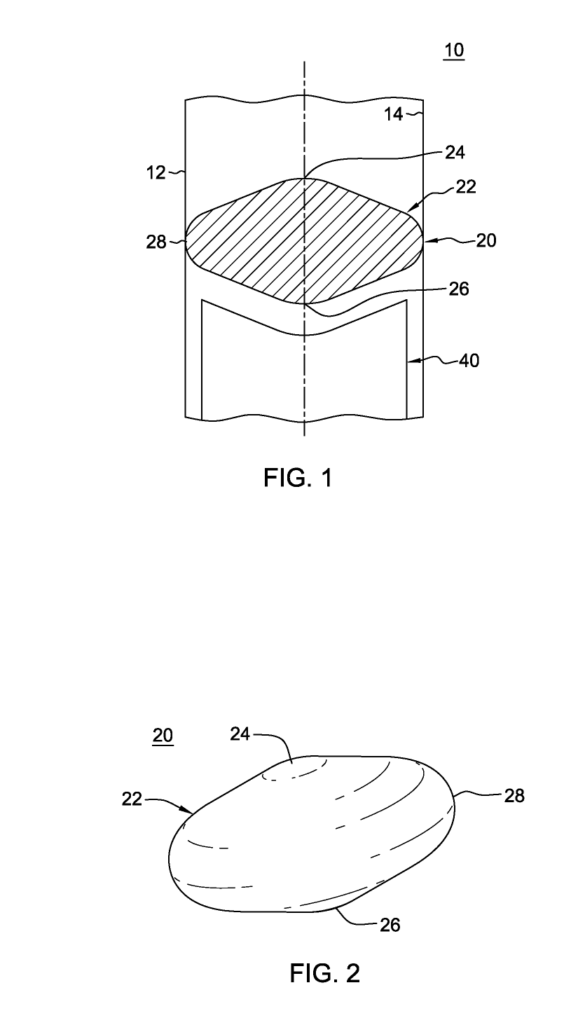

Referring to the drawings, wherein like reference numerals are used to indicate like or analogous components throughout the several views, and with particular reference to FIGS. 1-3, there is illustrated in one embodiment a syringe system 10 with a piston 20 and a plunger rod 40.

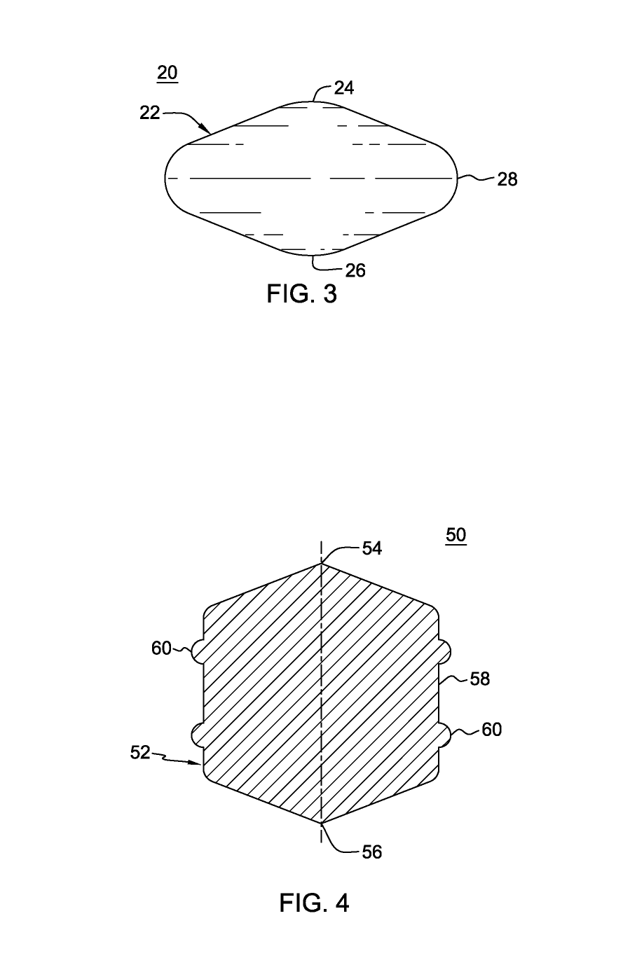

The piston 20, as shown in FIGS. 2 and 3, may include a base 22 with a first end 24, a second end 26 opposite the first end 24, and a side portion 28 extending between the first and second ends 24, 26. The first end 24 may be configured or shaped to engage, for example, a liquid medication inside of a syringe 12. The second end 26 may be configured or shaped to engage the plunger rod 40 for moving the piston 20. The side portion 28 may engage the interior surface 14 of the syringe 12, as seen in FIG. 1. The side portion 28, for example, may be configured as a rim, flange, ridge or planar surface for engaging the inner diameter of the syringe 12 to create a seal. The first end 24 and second end 26 may be, for example, symmetrical or asymmetrical.

The first and second ends 24, 26 may be symmetrical or mirror images of each other in order to allow for bi-directional installation. Bi-directional installation enables removal of the need to sort pistons using a vibratory piston bowl, which may in turn minimize the creation of particulate generation due to the vibrations. As shown in FIGS. 1-3, the first end 24 may be, for example, curved or planar in order to enable the piston 20 to conform to the geometry of the end of the syringe 12 to allow for consistent fluid injection into the patient. Further, the second end 26 may be, for example, curved or planar in order to self-center with respect to the end of the plunger rod 40, which may concentrate the force of the plunger rod 40 to the center of the piston 20. The first and second ends 24, 26 may also each have truncated cone shapes that are mirror images of each other. The configuration of the piston 20 may also increase the surface area contact between the wall of the syringe 12 and the piston 20. In addition, when the plunger rod 40 exerts an axial force onto the piston 20, because of the piston configuration the resultant forces move inward on impact from the plunger rod 40 and maintain the seal during stroke as well as minimize sidewall force and friction. The shape of the piston 20 allows for the outer diameter of the plunger rod 40 to be reduced by a third or more with respect to the inner diameter of the syringe 12.

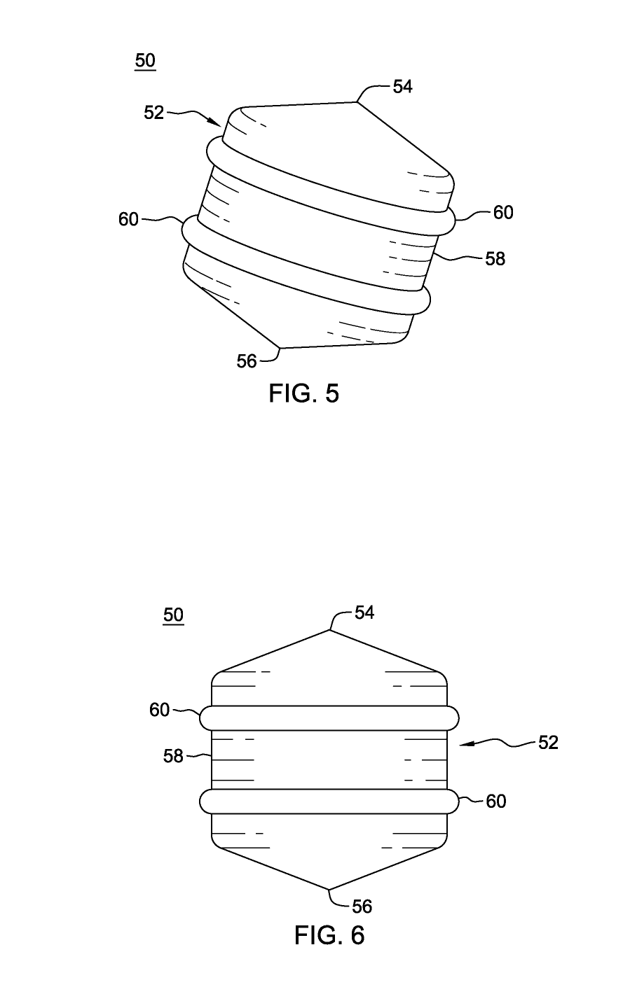

FIGS. 4-6 show another embodiment piston 50 including a base 52 with a first end 54, a second end 56, a side portion 58, and at least one engagement portion 60. The piston 50 may have a generally cylindrical cross sectional shape. The first end 54 may have, for example, a planar or curved surface that terminates at a pointed end or apex. The first end 54 may also be configured or shaped to contact a liquid medication inside of a syringe. The second end 56 may also have, for example, a planar or curved surface that terminates in a pointed end or apex. The second end 56 may be configured or shaped to engage a plunger rod, such as plunger rod 40, for moving the piston 50. In FIGS. 4-6, the piston 50 includes, for example, two circumferential engagement portions 60, such as extensions, rims, ridges, flanges, or protrusions, although the number of engagement portions 60 may be changed, for example, one to four engagement portions 60 depending on syringe size and medication type. The circumferential engagement portions 60 may be configured or shaped to engage an interior wall or an inner diameter of a syringe and may be configured or shaped to ensure a seal between the piston 50 and the interior surface of the syringe. The circumferential engagement portions 60 may also be positioned on the side portion 58 of the piston 50 to allow for minimal deformation during impact from a plunger rod. In addition, the engagement portions 60 may be, for example, integrated o-rings formed around the circumference of the side portion 58 of the piston 50.

The first end 54 and the second end 56 of the piston 50 may be configured or shaped to enable bi-directional installation of the piston 50. In addition, a plunger rod with a concave geometry, for example, the plunger rod 40 of FIG. 1, may be used with piston 50 to enable the piston 50 to self-center with respect to the plunger rod. When the plunger rod contacts the second end 56 of the piston 50, the at least one engagement portion 60 may deform to enable the piston 50 to start to slide along the interior surface of the syringe. As the piston 50 slides along the interior surface of the syringe to eject the medication, the dimensions of the at least one engagement portion 60 may normalize and a minimal sidewall friction will be exerted on the piston 50 as it moves along the length of the syringe.

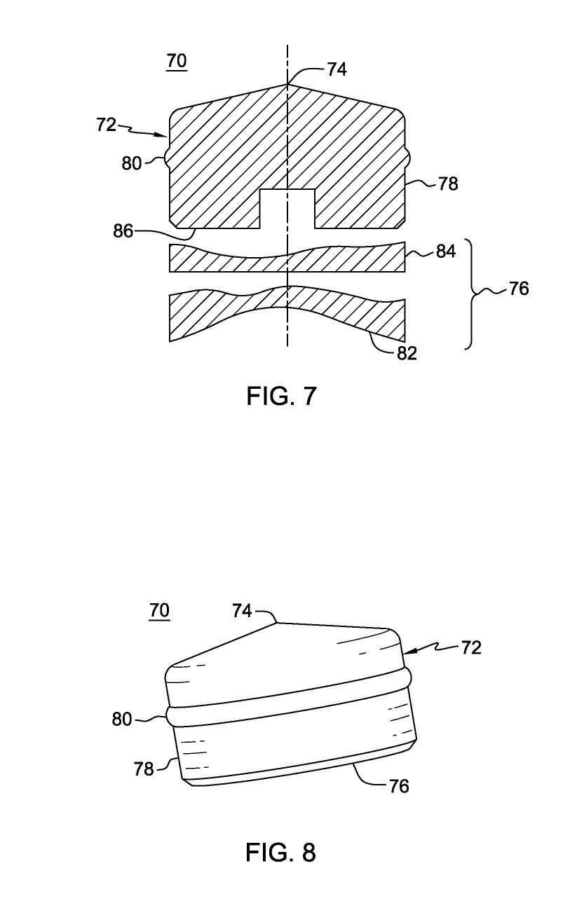

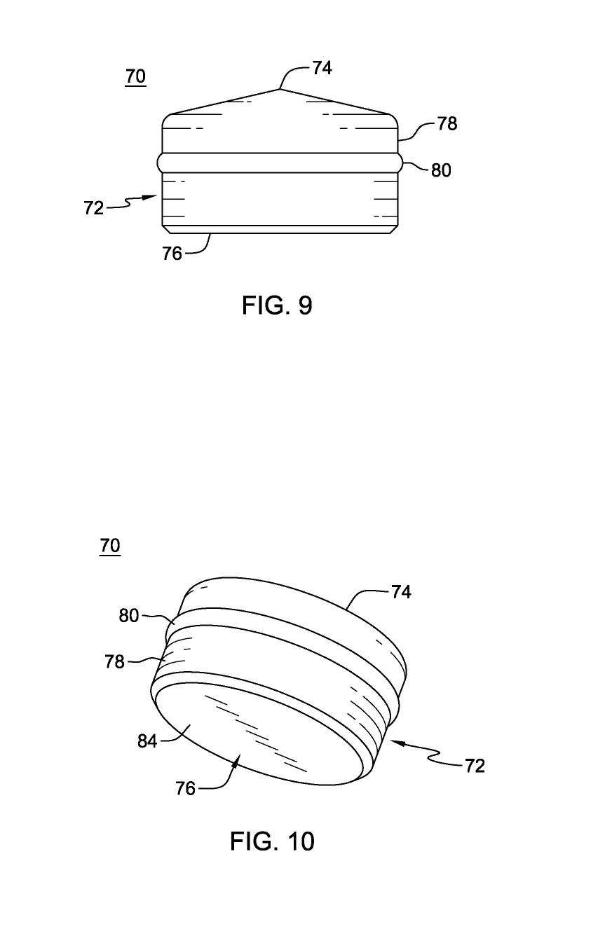

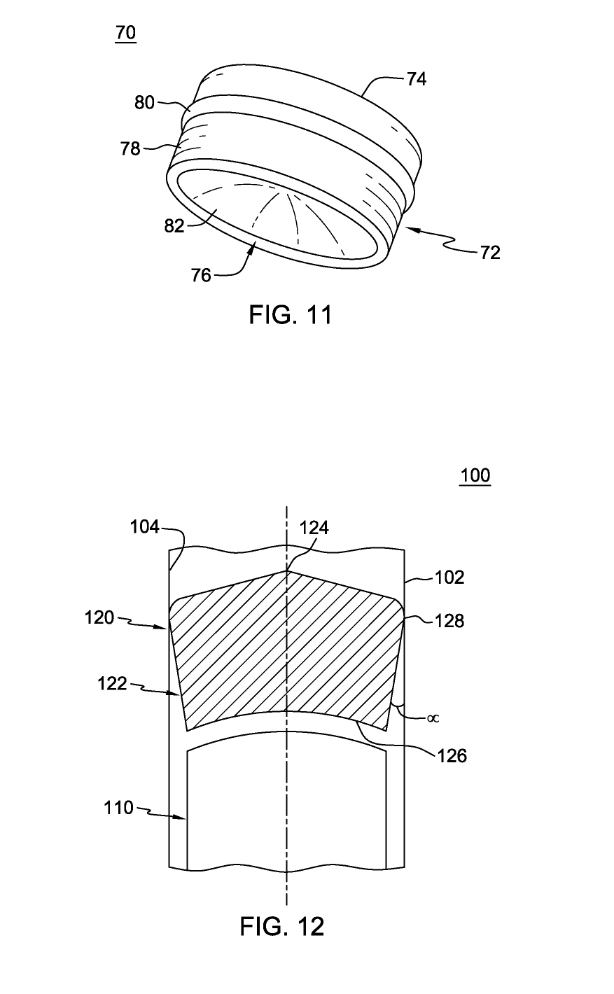

FIGS. 7-11 show another embodiment piston 70 including a base 72 with a first end 74, a second end 76 opposite the first end 74, a side portion 78 extending between the first and second ends 74, 76, and at least one circumferential engagement portion 80 on a surface of the side portion 78. The piston 70 may have a generally cylindrical cross-sectional shape. The first end 74 may be generally arcuate and configured or shaped to contact the medication inside of a syringe. The first end 74 may also be configured or shaped to conform to the syringe end of stroke geometry to ensure full dispensing of the medication. Full dispensing of the medication may be accomplished by shaping the first end 74 of the piston 70 to correspond to the shape of the interior surface of the injection end of the syringe. The second end 76 may be, for example, concave 82, planar 84, or notched 86 as shown in FIG. 7. The concave end 82 is also seen in FIG. 11 and the planar end 84 is also seen in FIG. 10. The second end 76 may also be configured or shaped to engage an end of a plunger rod, such as plunger rod 110 of FIG. 12, discussed in greater detail below, for moving the piston 70 within the syringe.

In FIG. 7, the piston 70 includes, for example, two circumferential engagement portions 80, while in FIGS. 8-11, the piston 70 includes, for example, one engagement portion 80, although other numbers of engagement portions 80 are also contemplated, for example, one to three engagement portions 80 may be used. The engagement portion(s) 80, for example a rim or extension, may be configured or shaped to slidingly contact an interior surface or an inner diameter of a syringe and may be configured or shaped to create a seal between the medication to be injected into the patient and the air in the syringe. The engagement portion 80 may also be positioned on the side portion 78 of the piston 70 to allow for minimal deformation following impact and movement of the plunger rod. In addition, the engagement portion 80 may be, for example, an integrated o-ring formed around the circumference of the side portion 78 of the piston 70. Alternatively, the engagement portion 80 may be, for example, helical around the side portion 78 and extending between at least a portion of the first and second ends 74, 76.

As a plunger rod (not shown) engages the second end 76 to dispense the medication from the syringe, the plunger rod contacts the center of the second end 76 first and compresses the center of the second end 76 causing the outer material from the side portion 78 to deform inward thereby reducing potential wall friction during the injection cycle. As the side portion 78 is deformed inward, the at least one engagement portion 80 may also be pulled inward reducing the friction between the inner surface or wall of the syringe and the engagement portion 80 of the piston 70 enabling consistent translation of the piston 70 along the longitudinal axis of the syringe.

FIG. 10 shows the piston 70 with a planar end 84. The planar end 84 would be contacted by the plunger rod to cause the piston 70 to move within the syringe for injection of a medication. As the planar end 84 is contacted by the piston 70, the planar end 84 may compress at the center pulling the outer material of the side portion 78 inward. In an alternative embodiment, as shown in FIG. 11, the piston 70 may include a concave end 82. As the concave end 82 is contacted by the piston 70, the concave end 82 may compress at the center and pull the outer material of the side portion 78 inward. The concave end 82 may allow for greater inward deformation of the piston 70 than the planar end 84.

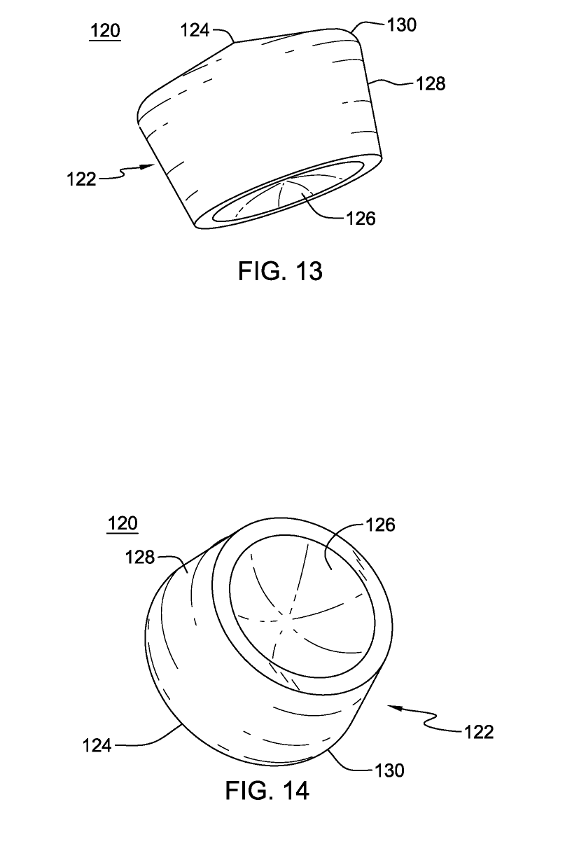

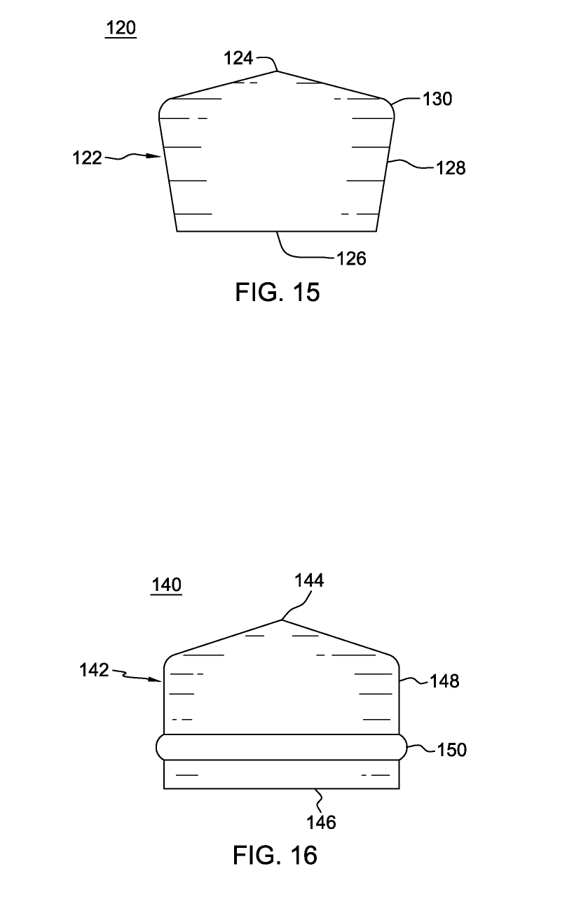

FIGS. 12-15 show yet another embodiment of syringe system 100 with a plunger rod 110 and a piston 120. The piston 120, as shown in FIGS. 12-15, may include a base 122 with a first end 124, a second end 126 opposite the first end 124, and a side portion 128 extending between the first and second ends 124, 126. The first end 124 may be configured or shaped to make contact with a medication located inside the cylinder of the syringe 102 for injection into a patient. The second end 126 may be configured or shaped to engage the head of the plunger rod 110 in order to move the piston 120 inside the syringe 102. The side portion 128 may be configured or shaped to engage the interior surface or wall 104 of the syringe 102, as seen in

FIG. 12. The side portion 128 may include an engagement portion 130 configured or shaped to correspond to the inner diameter of the syringe 102. The engagement portion 130 of the side portion 128 which contacts the syringe sidewalls after assembly and prior to use may have a height, for example, ranging from approximately 0.02″ to 0.06″ and more preferably range from about 0.03″ to about 0.05″. The piston 120 may have a modified trapezoid shape which may include a slightly concave second end 126. The modified trapezoid shape of the piston 120 may have the wider portion at the first end 124 and the narrower portion at the second end 126. The wider portion at the first end 124 makes contact with the interior syringe sidewalls to prevent leakage of the medication prior to and during injection. The side portion 128 may be angled at an angle a from the engagement portion 130 to the second end 126. The angle α may range from, for example, about 5° to 35° and more preferably from about 10° to about 30°.

As shown in FIGS. 12, 13 and 15, the first end 124 may be, for example, angled or curved in order to enable the piston 120 to conform to the geometry of the end of the syringe 102 to ensure that all medication is expelled. Further, the second end 126 may be, for example, curved inward into the base 122 of the piston 120. The second end 126 may be curved in order to allow for the concentration of force from the end of the plunger rod 110 into the center of the piston 120 resulting in a uniform force being applied during injection. The end of the plunger rod 110 may have a shape corresponding to the shape of the second end 126 of the piston 120. As force is applied by the plunger rod 110 to the second end 126 of the piston 120, the side portion 128 may deform to optimize the length of contact and degree of friction between the side portion 128 and the inner wall 104 of the syringe 102. The angle of deflection of the outer portion of the piston 120 is determined by the force and deformation at impact and during the stroke length.

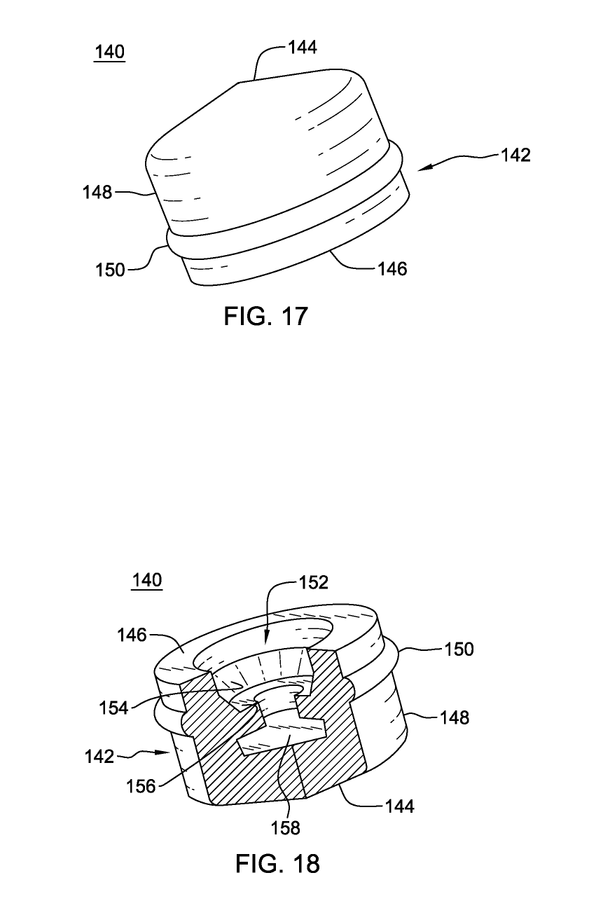

FIGS. 16-18 show yet another embodiment piston 140 including a base 142 with a first end 144, a second end 146 opposite the first end 144, a side portion 148 extending between the first and second ends 144, 146, and at least one circumferential engagement portion 150, for example a ridge or flange. The piston 140 may have a generally cylindrical cross-sectional shape. The first end 144 may be generally arcuate or angled and configured or shaped to contact the medication inside the container of the syringe. The first end 144 may also be configured or shaped to conform to the syringe end of stroke geometry to ensure the medication is fully dispensed.

The second end 146 may include, for example, an opening 152 as shown in FIG. 18. The opening 152 may include, for example, a tapered surface, angled or curved segment 154, a lip portion 156, and a central cavity or bore 158. The terms “tapered surface,” angled segment” and “curved segment” may be used interchangeably herein as they describe the same surface or segment of the opening 152. The opening 152 in the second end 146 is configured or shaped to engage the end of a plunger rod, such as plunger rod 110 of FIG. 12, or a similar configured end for moving the piston 140. The tapered surface 154 enables the side portion 148 to pull inward or collapse as the plunger rod 110 contacts the lip portion 156 and/or central cavity or bore 158 of the piston 140 during injection. As the side portion 148 moves inward after contact with the plunger rod the force between the side portion 148 of the piston 140 and the interior walls of the syringe reduces and enables the piston to break loose from its position and slide within the syringe to inject the medication. As the piston 140 slides within the syringe the configuration of the opening 152 allows for a low force friction to be maintained through the injection cycle.

In the piston embodiment 140 seen in FIGS. 16-18, there is one circumferential engagement portion 150, although other numbers of engagement portions 150 are also contemplated, for example, the piston 140 may include approximately one or five engagement portions 150. The engagement portion 150 will slidingly contact an interior surface or an inner wall of a syringe and may be configured or shaped to ensure a seal is formed between the medication to be injected into the patient and the air in the syringe. The engagement portion 150 may be positioned on the side portion 148 of the piston 140. The side portion 148 may deform inward when the force is applied to a plunger rod to engage the piston 140. As the side portion 148 deforms inward, the force to break the piston 140 loose for ejecting the medication is reduced and the piston 140 begins to slide along the interior wall of the syringe. As the piston 140 moves along the stroke length, a low friction force is maintained throughout the entire injection cycle. In addition, as the piston 140 moves, the engagement portion 150 maintains the seal integrity between the medication in one end of the syringe and the air in a second end of the syringe. The engagement portion 150 may be, for example, an integrated o-ring formed around the circumference of the side portion 148 of the piston 140. Alternatively, the engagement portion 150 may be, for example, helical around an exterior surface of the side portion 148. A helical engagement portion 150 may start near the first end 144 of the side portion 148 and spiral circumferentially around the side portion 148 moving toward the second end 146.

The plunger rod may be configured or shaped to have a corresponding end shape to the opening 152. As the end of the plunger rod engages the opening 152 of the second end 146 to dispense the medication, the plunger rod contacts the central cavity or bore 158 and compresses the center of the piston 140 causing the outer wall of the side portion 148 to be deformed inward thereby reducing potential sidewall friction during the injection cycle. As the side portion 148 is deformed inward, the at least one engagement portion 150 may also be deformed inward optimizing the amount of friction between the inner wall surface of the syringe and the engagement portion 150 of the piston 140 while still maintaining the seal integrity.

Each of the above described pistons 20, 50, 70, 120, and 140 may be incorporated into a syringe system, such as the syringe systems 10 or 100, as seen in FIGS. 1 and 12, respectively.

The syringe systems may include a plunger rod, such as plunger rod 40 or 110, for engaging a distal end of the pistons 20, 50, 70, 120, and 140. The syringe systems may also include plunger rods with proximal ends shaped to correspond to the distal ends of the pistons 20, 50, 70, 120, and 140, for example, concave, convex, planar, and the like. In addition, the pistons 20, 50, 70, 120, and 140 are configured or shaped to minimize side-wall friction variability to allow for a stabilized drug delivery and injection time. The pistons 20, 50, 70, 120, and 140 may also have a reduced body deflection when the plunger rods come into contact with the pistons 20, 50, 70, 120, and 140. The pistons 20, 50, 70, 120, and 140 are also all configured or shaped to decrease production and manufacturability costs and complexity, as well as, increase the dimensional consistency of the critical dimensions of the pistons 20, 50, 70, 120, and 140.

By way of specific example, if a 0.5 ml syringe with about a 4.65 mm inner diameter is used, the pistons 20, 50, 70, 120, and 140 may have an outer diameter of, for example, about 4.80 mm to about 5.10 mm and more preferably about 4.95 mm. If a 1 ml syringe with about a 6.35 mm inner diameter is used, the pistons 20, 50, 70, 120, and 140 may have an outer diameter of, for example, about 6.50 mm to about 6.80 mm and more preferably about 6.65 mm. If a 2 ml or 2.25 ml syringe with about a 8.65 mm diameter is used, the pistons 20, 50, 70, 120, and 140 may have an outer diameter of, for example, about 8.80 mm to about 9.10 mm and more preferably about 8.95 mm.

Further, the pistons 20, 50, 70, 120, and 140 may be made of, for example, butyl rubber or another elastic material. The pistons 20, 50, 70, 120, and 140 may also be coated with, for example, a bio-compatible coating, on at least the side of the pistons 20, 50, 70, 120, and 140 which contact the medication for injection.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

Having thus described the preferred embodiments, the invention is now claimed to be:

1. A piston for a syringe, comprising:
a base with a first end, a second end opposite the first end, and a side portion extending between the first end and the second end; and
at least one engagement portion extending around the circumference of the side portion of the base.

2. The piston of claim 1, wherein the first end is a mirror image of the second end.

3. The piston of claim 1, wherein the first end is arcuate from the side portion to a tip.

4. The piston of claim 1, wherein the first end is angled from the side portion to a tip.

5. The piston of claim 1, wherein the at least one engagement portion includes:
a first ridge extending around the circumference of the side portion of the base; and
a second ridge extending around the circumference of the side portion of the base, wherein the first ridge is spaced apart from the second ridge; and
the first ridge and second ridge are dimensioned to engage the inner diameter of the syringe.

6. The piston of claim 1, wherein the at least one engagement portion extends helically around at least a section of the side portion of the base between the first end and the second end.

7. The piston of claim 1, wherein the second end is planar.

8. The piston of claim 1, wherein the second end is concave.

9. The piston of claim 1, further comprising:
an opening for receiving an end of a plunger rod, wherein the opening comprises a tapered surface mating with a central cavity.

10. The piston of claim 9, wherein the opening further comprises a lip portion positioned intermediate the tapered surface and the central cavity.

11. The piston of claim 1, wherein the first end is shaped to contact a liquid medication and the second end is shaped to engage an end of a plunger rod of the syringe.

12. A plunger head for a syringe, comprising:
a proximal end;
a distal end opposite the proximal end; and
an intermediate portion connecting the proximal end and the distal end, wherein the intermediate portion is tapered from the proximal end to the distal end.

13. The plunger head of claim 12, further comprising:
at least one engagement portion extending around the circumference of the intermediate portion, the at least one engagement portion extending outwardly from the circumference of the intermediate portion to pressingly engage a side of a syringe chamber.

14. The plunger head of claim 13, wherein a top surface is curved from the at least one engagement portion to an apex at the proximal end.

15. The plunger head of claim 13, wherein the distal end has a concave surface.

16. A syringe system, comprising:
a piston with a proximal end, a distal end, a side section extending between the proximal end and the distal end, and at least one engagement portion extending around the circumference of the side section of the base and away from the side section, wherein the proximal end is shaped for engagement with a medication in a syringe chamber and the engagement portion is dimensioned to engage the syringe chamber; and
a plunger rod with a first end and a second end, wherein the first end pressingly contacts the distal end of the piston.

17. The syringe system of claim 16, wherein the engagement portion of the piston is configured to slidably contact an inner surface of the syringe chamber.

18. The syringe system of claim 16, wherein the first end of the plunger rod has a concave shape.

19. The syringe system of claim 16, wherein the distal end comprises:
an angled segment extending from the distal end into the base; and
a lip portion positioned intermediate the angled segment;
wherein the angled segment and lip portion form a bore for receiving the plunger rod.

20. The syringe system of claim 16, wherein the at least one engagement portion spirals around at least a portion of the side section of the base along a length of the base between the proximal end and the distal end.
